(19) United States
(12) Patent Application Publication
(10) Pub. No.: US 2022/0080006 A1
(43) Pub. Date: Mar. 17, 2022

(54) PROCESS FOR THE PREPARATION OF EASY-TO-TAKE TABLETS CONTAINING DRY EXTRACT OF GINKGO BILOBA LEAVES

(71) Applicant: DR. WILLMAR SCHWABE GMBH & CO. KG, Karlsruhe (DE)

(72) Inventors: Joachim HERRMANN, Karlsruhe (DE); Andreas Rothe, Karlsruhe (DE)

(73) Assignee: DR. WILLMAR SCHWABE GMBH & CO. KG, Karlsruhe (DE)

(21) Appl. No.: 17/422,259

(22) PCT Filed: Jan. 8, 2020
(86) PCT No.: PCT/EP2020/050306

§ 371 Date: Jul. 12, 2021

(30) Foreign Application Priority Data
Jan. 15, 2019 (EP) 19151878.6

(51) Int. Cl. A61K 36/16; A61K 9/00; A61K 9/20; A61K 9/28

(52) U.S. Cl. CPC A61K 36/16; A61K 9/0056; A61K 9/2095; A61K 9/2866; A61K 9/2059; A61K 9/2027; A61K 9/2013; A61K 9/2054

(57) ABSTRACT

A process prepares a rapidly disintegrating tablet with a disintegration time of at most 15 minutes for the peroral administration of a dry extract of the leaves of Ginkgo biloba, and with a total weight of the tablet of between 150 mg and 300 mg per 100 mg of ginkgo extract contained. Rapidly disintegrating tablets containing dry extract of the leaves of Ginkgo biloba can be prepared, which, due to their smaller dimensions, are easier to take than the tablets used hitherto. In a preferred form, the tablets do not contain lactose and are therefore also well tolerated.

The present invention relates to a process for the preparation of a rapidly disintegrating tablet with a disintegration time of at most 15 minutes for the peroral administration of a dry extract of the leaves of Ginkgo biloba and with a total weight of the tablet of between 150 mg and 300 mg per 100 mg of ginkgo extract contained. It is also an object of the invention to provide rapidly disintegrating tablets containing dry extract of the leaves of Ginkgo biloba, which, due to their smaller dimensions, are easier to take than the tablets used hitherto. In a preferred embodiment, the tablets do not contain lactose and are therefore also well tolerated.

Extracts of the leaves of Ginkgo biloba have been used as medicines for decades. Currently, they are used to treat various types of dementia and their symptoms as well as cerebral and peripheral circulatory disorders, tinnitus and dizziness. Ingredients with which efficacy is linked are terpene lactones (ginkgolides A, B, C and bilobalide) and glycosides of flavones (quercetin, kaempferol and isortiamnetin). According to Ph. Eur., medicinally used ginkgo dry extract contains 22.0 to 27.0% flavonoids calculated as flavone glycosides, 2.6 to 3.2% bilobalide, 2.8 to 3.4% ginkgolides A, B and C and at most 5 ppm ginkgolic acids. According to the European Pharmacopoeia, dry extracts generally have a loss on drying of not more than 5% by weight corresponding to a dry residue of not less than 95% by weight. The special extract EGb761@ contained in Tebonin® also meets this specification. These dementing diseases predominantly affect people of advanced age, who often have to take a variety of different medicines and suffer from swallowing difficulties due to the decreasing production of salivary fluid with age and often the presence of certain additional neurological diseases, such as Parkinson's disease. These problems in taking medicines often lead to reduced compliance to therapy due to medicines not being taken and, as a result, the failure of the medication to be successful.

According to the definition of the European Pharmacopoeia 9th edition 2017 (Ph.Eur.), tablets are solid medicinal preparations containing a single dose of one or more active ingredients. Tablets (uncoated) shall comply with the European Pharmacopoeia test 2.9.1. ‘Disintegration time’ and disintegrate within 15 minutes under the test conditions. To improve stability, ensure distinctness and improve swallowability, tablets are usually coated with a coloured film. If the film is a very thin polymer coating, the tablets are called film-coated tablets. Film-coated tablets must disintegrate within 30 minutes according to Ph.Eur.

The advantages of tablets, such as accurate dosage, are also countered by disadvantages, e.g. the need for them to be swallowed unchewed as a solid foreign body and to disintegrate within 15 minutes in order to exert their effect. A small tablet size and a short disintegration time are thus important properties. Achieving both can be problematic because tablets are manufactured by compressing a defined volume of particles and small, hard-pressed tablets with a high percentage by mass of drug inevitably have a longer disintegration time due to the physics involved than larger tablets that contain a higher percentage by mass of excipients such as disintegration accelerators and fillers and a smaller percentage of drug. A small tablet size in relation to the amount of active ingredient contained is more advantageous the larger the amount of active ingredient contained. Particular relevance therefore exists in the case of the 240 mg tablet, the one on the market with the highest active ingredient content. Should the provision of tablets with more than 240 mg ginkgo extract become necessary in the future, the availability of compact tablets will become even more important.

In the Red List (online edition 2017), 35 different products can be found for the active ingredient ginkgo. Of these, 28 are mono-products containing leaf extracts and the remaining seven are homeopathic medicinal products containing tinctures or dilutions prepared according to homeopathic rules. Among the dosage forms of the ginkgo leaf extract products, film tablets predominate with 25 products, and there are also three products as liquids (drops). The film-coated tablets contain 30, 40, 50, 60, 80, 120 or 240 mg of dry extract of the leaves of Ginkgo biloba. All products in the solid dosage form “film-coated tablet” listed in this German Pharmacopoeia and known to date contain the ingredient lactose or lactose monohydrate as part of the composition, as can be seen from the product documentation.

Table M below lists the products with 240 mg dry extract of ginkgo leaves sold on the market in Germany, their dimensions and weight. According to the respective package leaflet, all products contain lactose. All the tablets mentioned are rapidly disintegrating film-coated tablets with a disintegration time of 30 minutes or less.

TABLE 1
Dimensions and weights of the products currently sold in Germany

| | | | Ratio of the mass | |
|---|---|---|---|---|
| | Dimensions | Mass of tablet | of the tablet to | Mass of the |
| | Length/width/thickness | without coating* | the mass of the | coated tablet |
| Product designation | [mm] | [mg] | active ingredient | [mg] |
| Binko 240 | 19, 2/8, 1/6, 1 | At least 781* | 3, 25 | 839 |
| Doppelherz ® Ginkgo 240 | 19, 3/8, 2/6, 3 | At least 780* | 3, 25 | 837 |
| Gingobeta 240 | 19, 2/8, 1/6, 0 | At least 796* | 3, 32 | 846 |
| Ginkgo AL 240 | 19, 2/8, 1/6, 1 | At least 768* | 3, 20 | 824 |
| Gingium ® extra 240 | 20, 2/9, 2/5, 8 | At least 780* | 3, 25 | 814 |
| Ginkgovital ® Neumann 240 | 19, 3/8, 2/6, 1 | At least 793* | 3, 30 | 844 |
| Ginkgo maren ® 240 | 19, 2/9, 0/5, 6 | At least 811* | 3, 38 | 855 |
| Ginkgo STADA ® 240 | 19, 2/8, 1/6, 1 | At least 784* | 3, 27 | 838 |
| Ginkobii ® ratiopharm 240 | 20, 2/9, 2/5, 8 | At least 780* | 3, 25 | 814 |
| Tebonin ® konzent 240 | 19, 2/8, 2/6, 0 | 780 | 3, 25 | 814 |

*Measured on tablet cores freed from coating by peeling off.

The mass fraction of active ingredient in the total mass of the non-coated tablet is between 31.3 and 29.6% for the tablets on the market in Germany, or the ratio of tablet mass to contained active ingredient mass (TM/WM) is 3.20-3.38 corresponding to a tablet mass of 320 mg to 338 mg per 100 mg of ginkgo extract contained.

EP2072054A1 describes a tablet with 240 mg dry extract of Ginkgo biloba leaves and a mass of the tablet core of 800 mg (i.e. with a TM/WM=3.33 corresponding to a tablet mass of 333 mg per 100 mg of Ginkgo extract contained), 160 mg of which is lactose monohydrate (see Comparative Example 1 and EP2072054A1, Example 3 according to the invention). The tablet described is a rapid-release tablet (EP2072054A1, Example 3 “Conventional-release dosage form” according to the invention and claim 15).

Lactose is a disaccharide found in milk consisting of galactose and glucose. In its anhydrous form, lactose is hygroscopic; from the aqueous solution, the more stable α-form crystallises out as a monohydrate. Lactose is the most commonly used basic substance for tablets (“Die Tablette”; W. A. Ritschel, A. Bauer-Brandl; ECV Verlag Aulendorf, 2002, p. 74). In lactose intolerance, the lactose ingested with food is not or incompletely digested as a result of missing or reduced production of the digestive enzyme lactase. The lactose that is not digested in the small intestine reaches the large intestine in lactose-intolerant people and is fermented there as a nutrient by the intestinal flora. The result is mainly flatulence, abdominal pressure, abdominal cramps, nausea, vomiting and often spontaneous diarrhoea.

WO2012/146592A1 (granted as EP2701688B1) describes a tablet with controlled release containing 240 mg of dry extract of Ginkgo biloba leaves and its preparation (see comparative example 2). This tablet is lactose-free, weighs 420 mg, contains very few excipients, has an active ingredient content of 57% m/m (TM/WM=1.75 corresponding to a tablet mass of 175 mg per 100 mg of the ginkgo extract contained) and is thus very compact. However, according to the definition of the European Pharmacopoeia, this embodiment is a tablet with altered release of the active ingredient, in this case a so-called retard tablet, in which the disintegration of the tablet is deliberately slowed down and the active ingredient is released in a controlled manner over a period of more than six hours.

Compared to the fast-releasing compact and lactose-free tablets according to the invention and the fast-releasing large and lactose-containing tablets described in EP2072054A1, the retard tablet described in WO2012/146592A1 (granted as EP2701688B1) has the disadvantage that the active ingredient is absorbed more slowly into the body and becomes effective later. So far, no retard tablet with the active ingredient ginkgo leaf extract has been launched on the market in Germany because the release behaviour of this tablet differs from all other products and therefore the efficacy still has to be proven in expensive clinical trials.

The special, controlled release of the active ingredient in the retard tablet is achieved by the choice of excipients according to type and quantity, i.e. by using a small amount of excipients, by using water-insoluble ethyl cellulose with low swelling capacity as a retarding agent and by not using disintegration accelerators such as croscarmellose sodium.

DE 20 2014 005 450 U1 describes a lactose-free medicinal product containing a dry extract from artichoke leaves. The embodiment is a solid pharmaceutical dosage form in the form of a hard capsule or a tablet. Although this document provides information on the qualitative composition of the lactose-free medicinal product, it does not provide information on how the proper preparation of this form is achieved and how large or heavy the prepared pharmaceutical form is.

The task was to provide a process for the preparation of a rapidly disintegrating tablet with a disintegration time of at most 15 minutes for the peroral administration of a dry extract of the leaves of Ginkgo biloba and with a total weight of the tablet of between 150 mg and 300 mg per 100 mg of Ginkgo extract contained. The task was also to provide rapidly disintegrating tablets with dry extract of the leaves of Ginkgo biloba with small dimensions, i.e. good swallowability and, in a preferred embodiment, without lactose, i.e. with good tolerability.

The dry extract of Ginkgo biloba leaves is a very fine, brown coloured powder which, when stored in the open, absorbs moisture from the environment very quickly and tends to stick together. Due to the small particle size of at least 90%<100 μm and the rapid and strong moisture absorption, mixtures of this extract with the usual pharmaceutical excipients have poor flow properties and are poorly suited for the production of tablets by direct compression. For this reason, excipients are needed that improve the flowability of the extract and enable the production of a tablet by compressing the powder mixture. An ideal excipient to improve the flowability and compressibility is lactose monohydrate. For this reason, lactose is included in almost all high-dose products containing ginkgo leaf extract.

Surprisingly, it has now been possible to produce a concentrated granulate (compactate) by reducing the specific surface area of the ginkgo extract by adding a small proportion of the excipients contained in the tablet and compacting this mixture by means of compaction and subsequent comminution (step (a); low proportion of excipients in this context means that the compactate mass is 1.14 to 1.57 times the active ingredient mass). This compacted granulate has such good flowability that no lactose is needed to achieve the flow properties required for tablet production (see comparative example 1).

By mixing this granulate with further pharmaceutically usual excipients, i.e. microcrystalline cellulose (binder), croscarmellose sodium, carboxymethyl starch sodium, crospovidone or sodium starch glycolate (disintegration accelerator), highly dispersed silica or precipitated silica (flow regulating agent) and magnesium stearate, stearic acid, behenic acid, sodium stearyl fumarate, glycerol dibehenate, calcium behenate or fumaric acid (mould release agent), tablets are obtained (step (b)) which, despite the high proportion of active ingredient and the low proportion of excipients, have a very short disintegration time (see Table 2), which is even lower than that of the tablets from Comparative Example 1.

Thus, it is an object of the invention to provide a process for the preparation of a rapidly disintegrating tablet having a disintegration time of at most 15 minutes for the peroral administration of a dry extract of the leaves of Ginkgo biloba and having a total weight of the tablet of between 150 mg and 300 mg per 100 mg of ginkgo extract contained (corresponding to a total weight of between 360 mg and 720 mg in the case of the tablet containing 240 mg of ginkgo extract), comprising
(a) a first process step in which the ginkgo extract is mixed with 6.94 mg to 27.78 mg of binder per 100 mg of ginkgo extract contained, with 5.56 mg to 22.22 mg of disintegration accelerator per 100 mg of ginkgo extract contained, with 1.11 mg to 4.44 mg of flow regulating agent per 100 mg of ginkgo extract contained and with 0.28 mg to 1.11 mg of mould release agent per 100 mg of ginkgo extract contained and then compacted and comminuted to obtain a concentrated granulate (compactate), and
(b) a second process step in which the concentrated granulate obtained in step (a) is mixed with a further 22.78 mg to 91.11 mg of binder per 100 mg of ginkgo extract contained, with a further 11.11 mg to 44.44 mg of disintegration accelerator per 100 mg of ginkgo extract contained, with a further 0.56 mg to 2.22 mg of flow regulating agent per 100 mg of ginkgo extract contained and with a further 1.67 mg to 6.67 mg of mould release agent per 100 mg of ginkgo extract contained and pressed into tablets,
   wherein the total amount of excipients in step (a) is from 13.89 mg to 55.56 mg per 100 mg of ginkgo extract contained and in step (b) is from 36.11 mg to 144.44 mg per 100 mg of ginkgo extract contained; and
   wherein the binder in step (a) and in step (b) is microcrystalline cellulose, the disintegration accelerator in step (a) and in step (b) is independently selected from croscarmellose sodium, carboxymethyl starch sodium, crospovidone or sodium starch glycolate, the flow regulating agent in step (a) and in step (b) is independently selected from highly dispersed silica or precipitated silica, and the mould release agent in step (a) and in step (b) is independently selected from magnesium stearate, stearic acid, behenic acid, sodium stearyl fumarate, glycerol dibehenate, calcium behenate or fumarcacid.

Preferred is a process for preparing a rapidly disintegrating tablet having a disintegration time of at most 15 minutes for the peroral administration of a dry extract of the leaves of Ginkgo biloba and having a total weight of the tablet of between 160 mg and 200 mg per 100 mg of ginkgo extract contained (corresponding to a total weight of between 384 mg and 480 mg in the case of the tablet containing 240 mg of ginkgo extract), comprising
(a) a first process step in which the ginkgo extract is mixed with 8.33 mg to 13.89 mg of binder per 100 mg of ginkgo extract contained, with 6.67 mg to 11.11 mg of disintegration accelerator per 100 mg of ginkgo extract contained, with 1.33 mg to 2.22 mg of flow regulating agent per 100 mg of ginkgo extract contained and with 0.33 mg to 0.56 mg of mould release agent per 100 mg of ginkgo extract contained and then compacted and comminuted to obtain a concentrated granulate (compactate), and
(b) a second process step in which the concentrated granulate obtained in step (a) is mixed with a further 27.33 mg to 45.56 mg of binder per 100 mg of ginkgo extract contained, with a further 13.33 mg to 22.22 mg of disintegration accelerator per 100 mg of ginkgo extract contained, with a further 0.67 mg to 1.11 mg of flow regulating agent per 100 mg of ginkgo extract contained and with a further 2.00 mg to 3.33 mg of mould release agent per 100 mg of ginkgo extract contained and pressed into tablets,
   wherein the total amount of excipients in step (a) is 16.67 mg to 27.78 mg per 100 mg of ginkgo extract contained and in step (b) is 43.33 mg to 72.22 mg per 100 mg of ginkgo extract contained, and
   wherein the binder in step (a) and in step (b) is microcrystalline cellulose, the disintegration accelerator in step (a) and in step (b) is independently selected from croscarmellose sodium, carboxymethyl starch sodium, crospovidone or sodium starch glycolate, the flow regulating agent in step (a) and in step (b) is independently selected from highly dispersed silica or precipitated silica, and the mould release agent in step (a) and in step (b) is independently selected from magnesium stearate, stearic acid, behenic acid, sodium stearyl fumarate, glycerol dibehenate, calcium behenate or fumaric acid.

In a preferred embodiment of the above process, the disintegration accelerator in step (a) and in step (b) is croscarmellose sodium, the flow regulating agent in step (a) and in step (b) is precipitated silica, and the mould release agent in step (a) and in step (b) is magnesium stearate.

In another preferred embodiment of the above process, the compacting in step (a) is carried out by roller compacting.

In a further preferred embodiment of the above process, no plant extracts or other active ingredients are used apart from ginkgo extract and no other excipients are used apart from the excipients mentioned.

The excipients in step (a) of the process described above are composed of 45 wt % to 55 wt % binder, 36 wt % to 44 wt % disintegration accelerator, 7 wt % to 9 wt % flow regulating agent and 1.5 wt % to 2.5 wt % mould release agent. The excipient composition in step (b) is 57 wt % to 69 wt % binder, 28 wt % to 34 wt % disintegration accelerator, 1 wt % to 2 wt % flow regulating agent and 4 wt % to 5 wt % mould release agent.

It is further an object of the invention to provide a rapidly disintegrating tablet having a disintegration time of at most 15 minutes for peroral administration of a dry extract of the leaves of Ginkgo biloba and having a total weight of the tablet of between 150 mg and 300 mg per 100 mg of ginkgo extract contained (corresponding to a total weight of between 360 mg and 720 mg in the case of the tablet containing 240 mg of ginkgo extract), obtainable by the process described above.

In a preferred embodiment, the tablet is characterised in that it contains 80 to 360 mg of dry extract of the leaves of Ginkgo biloba.

In a particularly preferred embodiment, the tablet is characterised in that it contains 220 to 260 mg of dry extract of the leaves of Ginkgo biloba.

In an even more preferred embodiment, the tablet is characterised by containing 240 mg of dry extract of the leaves of Ginkgo biloba.

Preferably, the above tablets contain a dry extract of the leaves of Ginkgo biloba containing 22.0 to 27.0% flavonoids calculated as flavone glycosides, 2.6 to 3.2% bilobalide, 2.8 to 3.4% ginkgolides A, B and C and at most 5 ppm ginkgolic acids. The percentages refer to the weight (% by weight).

In another preferred embodiment, the tablets described above do not contain lactose.

In a further preferred embodiment, the tablets described above are additionally coated with a film and have a disintegration time of at most 30 minutes.

In addition, it is also an object of the invention to provide a rapidly disintegrating tablet having a disintegration time of at most 15 minutes for peroral administration of a dry extract of the leaves of Ginkgo biloba, characterised in that the total weight of the tablet is between 150 mg and 300 mg per 100 mg of ginkgo extract contained (corresponding to a total weight between 360 mg and 720 mg in the case of the tablet containing 240 mg of ginkgo extract), the tablet containing, in addition to the ginkgo extract, microcrystalline cellulose as a binder, a disintegration accelerator selected from croscarmellose sodium, carboxymethyl starch sodium, crospovidone or sodium starch glycolate, a flow regulating agent selected from highly dispersed silica or precipitated silica and a mould release agent selected from magnesium stearate, stearic acid, behenic acid, sodium stearyl fumarate, glycerol dibehenate, calcium behenate or fumaric acid.

Preferably, the total weight of the tablet is between 160 mg and 200 mg per 100 mg of ginkgo extract contained (corresponding to a total weight between 384 mg and 480 mg in the case of the tablet containing 240 mg of ginkgo extract).

Particularly preferably, the total weight of the tablet is 175 mg per 100 mg of ginkgo extract contained (corresponding to a total weight of 420 mg in the case of the tablet containing 240 mg of ginkgo extract).

In another preferred embodiment, the tablet contains 80 to 360 mg of dry extract of the leaves of Ginkgo biloba.

Particularly preferably, the tablet contains 220 to 260 mg of dry extract of the leaves of Ginkgo biloba.

Very preferably, the tablet contains 240 mg of dry extract of the leaves of Ginkgo biloba.

Preferably, the above tablets contain a dry extract of the leaves of Ginkgo biloba containing 22.0 to 27.0% flavonoids calculated as flavone glycosides, 2.6 to 3.2% bilobalide, 2.8 to 3.4% ginkgolides A, B and C and at most 5 ppm ginkgolic acids. The percentages refer to the weight (% by weight).

In another preferred embodiment, the tablets described above do not contain lactose.

In a further preferred embodiment, the tablets described above do not contain any plant extracts or other active ingredients other than ginkgo extract and no excipients other than the excipients mentioned.

Furthermore, the tablets described above preferably contain microcrystalline cellulose, croscarmellose sodium, precipitated silica and magnesium stearate as excipients.

In a further preferred embodiment, the tablets described above are additionally coated with a film and have a disintegration time of at most 30 minutes.

The quantities given in the above description, in the examples and in the claims have been obtained by proportional conversion of the composition in example 2 and by reference to 100 mg ginkgo extract and rounded to two decimal places. Possible inaccuracies in summations can therefore not be excluded.

### EXAMPLE 1 ACCORDING TO THE INVENTION

Compact, Lactose-Free Tablets without Coating Containing 240 mg Dry Extract of Ginkgo biloba Leaves with a Ratio of Tablet Mass to Active Ingredient Mass Contained of 1.5

| | | | Calculated on |
|---|---|---|---|
| | | | 100 mg of the |
| | | Amount | contained |
| | | per tablet | ginkgo extract |
| | Component | [mg] | [mg]. |
| 1. | Ginkgo leaf extract (EGb ® 761) | 240.00 | 100.00 |
| 2. | Cellulose, microcrystalline | 16.67 | 6.94 |
| 3. | Croscarmellose sodium | 13.33 | 5.56 |
| 4. | Silica, precipitated | 2.67 | 1.11 |
| 5. | Magnesium stearate | 0.67 | 0.28 |
| 2.-5. | Sum of excipients in step (a) | 33.34 | 13.89 |
| 1.-5. | Compactate | 273.34 | 113.89 |
| 6. | Cellulose, microcrystalline | 54.66 | 22.78 |
| 7. | Croscarmellose sodium | 26.67 | 11.11 |
| 8. | Silica, precipitated | 1.33 | 0.56 |
| 9. | Magnesium stearate | 4.00 | 1.67 |
| 6.-9. | Sum of excipients in step (b) | 86.66 | 36.11 |
| 1.-9. | Tablet | 360.00 | 150.00 |

For the preparation of the tablets according to the invention as described in Example 1, 480 g of ginkgo leaf extract are mixed with 33.34 g of microcrystalline cellulose, 26.66 g of croscarmellose sodium, 5.34 g of precipitated silica and 1.34 g of magnesium stearate and processed with a roller compactor to a concentrated granulate (compactate) (step (a)). To produce 1600 tablets, 437.34 g of the granulate thus obtained are mixed with 87.46 g of microcrystalline cellulose, 42.67 g of croscarmellose sodium, 2.13 g of precipitated silica and 6.40 g of magnesium stearate and compressed on a rotary tablet press to form tablets with a mass of 360 mg each (step (b)).

### EXAMPLE 2 ACCORDING TO THE INVENTION

Compact, Lactose-Free Tablets without Coating Containing 240 mg Dry Extract of Ginkgo biloba Leaves with a Ratio of Tablet Mass to Active Ingredient Mass Contained of 1.75

| | | | Calculated on |
|---|---|---|---|
| | | | 100 mg of the |
| | | Amount | contained |
| | | per tablet | ginkgo extract |
| | Component | [mg] | [mg]. |
| 1. | Ginkgo leaf extract (EGb ® 761) | 240.00 | 100.00 |
| 2. | Cellulose, microcrystalline | 25.00 | 10.42 |
| 3. | Croscarmellose sodium | 20.00 | 3.33 |
| 4. | Silica, precipitated | 4.00 | 1.67 |
| 5. | Magnesium stearate | 1.00 | 0.42 |
| 2.-5. | Sum of excipients in step (a) | 50.00 | 20.83 |
| 1.-5. | Compactate | 290.00 | 120.83 |
| 6. | Cellulose, microcrystalline | 82.00 | 34.17 |
| 7. | Croscarmellose sodium | 40.00 | 16.67 |
| 8. | Silica, precipitated | 2.00 | 0.83 |
| 9. | Magnesium stearate | 6.00 | 2.50 |
| 6.-9. | Sum of excipients in step (b) | 130.00 | 54.17 |
| 1.-9. | Tablet | 420.00 | 175.00 |

To produce 100,000 tablets of the invention according to Example 2, 24.00 kg of ginkgo leaf extract is mixed with 2.50 kg of microcrystalline cellulose, 2.00 kg of croscarmellose sodium, 0.40 kg of precipitated silica and 0.10 kg of magnesium stearate and processed with a roller compactor to form a concentrated granulate (compactate) (step (a)). The granulate is mixed with 8.20 kg microcrystalline cellulose, 4.00 kg croscarmellose sodium, 0.20 kg precipitated silica and 0.60 kg magnesium stearate and compressed on a rotary tablet press to form tablets with a mass of 420 mg each (step (b)).

### EXAMPLE 3 ACCORDING TO THE INVENTION

Compact, Lactose-Free Tablets without Coating Containing 240 mg Dry Extract of Ginkgo biloba Leaves with a Ratio of Tablet Mass to Active Ingredient Mass Contained of 3.0

| | | | Calculated on |
|---|---|---|---|
| | | | 100 mg of the |
| | | Amount | contained |
| | | per tablet | ginkgo extract |
| | Component | [mg] | [mg]. |
| 1. | Ginkgo leaf extract (EGb ® 761) | 240.00 | 100.00 |
| 2. | Cellulose, microcrystalline | 66.67 | 27.78 |
| 3. | Croscarrnellose sodium | 53.33 | 22.22 |
| 4. | Silica, precipitated | 10.67 | 4.44 |
| 5. | Magnesium stearate | 2.67 | 1.11 |
| 2.-5. | Sum of excipients in step (a) | 133.34 | 55.56 |
| 1.-5. | Compactate | 373.34 | 155.56 |
| 6. | Cellulose, microcrystalline | 218.66 | 91.11 |
| 7. | Croscarmellose sodium | 106.67 | 44.44 |
| 8. | Silica, precipitated | 5.33 | 2.22 |
| 9. | Magnesium stearate | 16.00 | 6.67 |
| 6.-9. | Sum of excipients in step (b) | 346.66 | 144.44 |
| 1.-9. | Tablet | 720.00 | 300.00 |

For the preparation of the tablets according to the invention as in Example 3, 480 g of ginkgo leaf extract are mixed with 133.34 g of microcrystalline cellulose, 106.66 g of croscarmellose sodium, 21.34 g of precipitated silica and 5.34 g of magnesium stearate and processed with a roller compactor to a concentrated granulate (compactate) (step (a)). To produce 1600 tablets, 597.34 g of the granulate thus obtained are mixed with 349.66 g of microcrystalline cellulose, 170.67 g of croscarmellose sodium, 8.53 g of precipitated silica and 25.60 g of magnesium stearate and compressed on a rotary tablet press to form tablets with a mass of 720 mg each (step (b)).

### EXAMPLE 4 ACCORDING TO THE INVENTION

Compact, Lactose-Free Tablet with Coating Containing 240 mg Dry Extract of Ginkgo biloba Leaves

The lactose-free, compact tablets of Example 2 according to the invention can be provided with a thin coloured coating which hardly delays the disintegration of the tablets and thus the release of the active ingredient (cf. Table 2).

| | | Mass fraction in mg |
|---|---|---|
| | Component | per film-coated tablet |
| 1. | Ginkgo leaf extract (EGb ® 761) | 240.00 |
| 2. | Cellulose, microcrystalline | 107.00 |
| 3. | Croscarmellose sodium | 60.00 |
| 4. | Silica, precipitated | 6.00 |
| 5. | Magnesium stearate | 7.00 |
| | Subtotal tablet weight without coating | 420.00 |
| 6. | Hypromellose | 4.80 |
| 7. | Microcrystalline cellulose | 2.00 |
| 8. | Glycerol, anhydrous | 0.70 |
| 9. | Iron oxide E172 | 3.00 |
| 10. | Talc | 1.50 |
| | Subtotal film coating weight | 12.00 |
| | Total film-coated tablet weight | 434.00 |

### NON-INVENTIVE COMPARATIVE EXAMPLES

### Comparative Example 1 from EP 2072054A1

EP2072054A1 describes the use of an extract from leaves of Ginkgo biloba in the preferred embodiment of a tablet with a mass of 800 mg, of which 160 mg is lactose monohydrate. For the production (paragraph [0033]) of the tablet according to Example 3 of EP 2072054A1 according to the invention, the extract is mixed with the excipients mentioned in the following table and pressed directly into tablets without further process steps. The tablet described is a tablet with rapid release of the active ingredient (EP2072054A1, claim 15 and table for Example 1 according to the invention). This embodiment is significantly larger and heavier than the tablet according to the invention and contains lactose monohydrate.

| | | | Calculated on |
|---|---|---|---|
| | | | 100 mg of the |
| | | Amount | contained |
| | | per tablet | ginkgo extract |
| | Component | [mg] | [mg]. |
| 1. | Ginkgo leaf extract EGb ® 761 | 240.00 | 100.00 |
| 2. | Cellulose, microcrystalline | 290.00 | 120.83 |
| 3. | Lactose monohydrate | 160.00 | 66.67 |
| 4. | Maize starch | 50.00 | 20.83 |
| 5. | Croscarrnellose sodium | 40.00 | 16.67 |
| 6. | Silica, highly dispersed | 10.00 | 4.17 |
| 7. | Magnesium stearate | 10.00 | 4.17 |
| | Tablet weight | 800.00 | 333.33 |

### Comparative Example 2 from EP 2701688B1

WO2012/146592A1 (granted as EP2701688B1) describes a tablet containing 240 mg of controlled release dry extract of Ginkgo biloba leaves and its preparation. The tablet is prepared as described in Example 1 of WO2012/146592A1 according to the invention. In this regard, the composition is shown in the table below. This tablet is lactose-free, contains very few excipients and is thus very compact. However, according to the definition of the European Pharmacopoeia, this embodiment is a tablet with modified release of the active ingredient, in this case a so-called retard tablet, in which the disintegration of the tablet is deliberately slowed down and the active ingredient is released in a controlled manner over a period of more than six hours (see table for Example 1 according to the invention).

| | | | Calculated on |
|---|---|---|---|
| | | | 100 mg of the |
| | | Amount | contained |
| | | per tablet | ginkgo extract |
| | Component | [mg] | [mg]. |
| 1. | Ginkgo leaf extract EGb ® 761 | 240.00 | 100.00 |
| 2. | Ethyl cellulose | 170.00 | 70.83 |
| 3. | Silica, highly dispersed | 2.00 | 0.83 |
| 4. | Magnesium stearate | 6.00 | 3.33 |
| | Tablet weight | 420.00 | 175.00 |

### Comparison of Disintegration Times and Dimensions

According to the specifications of the European Pharmacopoeia Ph.Eur. 2.9.1, the disintegration times of the compact tablets according to the invention according to Examples 1 to 4 and the tablets of Comparative Example 1 (large, fast-disintegrating tablet with lactose) and Comparative Example 2 (compact retard tablet) were determined.

TABLE 2
Disintegration times and dimensions of the tablets from Examples 1
to 4 according to the invention and Comparative Examples 1 and 2.

| | | | Disintegration | Dimensions |
|---|---|---|---|---|
| | Batch | | time, | Length/Width/Thickness |
| Embodiment | designation | TM/WM* | n = 6 [min] | [mm] |
| Compact tablet according | 201804 | 1.50 | 5:14-7:00 | 14.46/7.56/4.13 |
| to the invention (without | | | | |
| coating) according to | | | | |
| example 1 | | | | |
| Compact tablet according | P201602 | 1.75 | 7:18-9:32 | 14.08/7.07/5.18 |
| to the invention (without | | | | |
| coating) according to | | | | |
| example 2 | | | | |
| Compact tablet according | 201805 | 3.00 | 4:12-5:04 | 17.14/8.08/6.97 |
| to the invention (without | | | | |
| coating) according to | | | | |
| example 3 | | | | |
| Compact film-coated tablet | P201602 | 1.75 | 9:14-11:16 | 14.06/7.09/5.28 |
| according to the invention | | (without | | |
| (with coating) according to | | coating) | | |
| example 4 | | | | |
| Tablet (without coating) | P201301 | 3.33 | 10:20-13:00 | 19.15/8.07/5.82 |
| according to comparative | | | | |
| example 1 | | | | |
| Retard tablet (without | 201401 | 1.75 | >30 min | round tablet |
| coating) according to | | | | (11 mm) |
| comparative example 2 | | | | |

*TM/WM = ratio tablet mass/active ingredient mass

The tablets according to the invention have a short disintegration time of less than 15 minutes (without coating, examples 1 to 3) or of less than 30 minutes (with coating, example 4) despite a high active ingredient content or the low tablet mass in relation to the active ingredient mass contained and thus comply with the specifications of the European Pharmacopoeia.

Thereby, the tablets of examples 1 to 4 according to the invention are smaller than the tablets of comparative example 1 with the same active ingredient content.

1-17. (canceled)

18: A process for the preparation of a rapidly disintegrating tablet having a disintegration time of at most 15 minutes for the peroral administration of a dry extract of leaves of Ginkgo biloba and having a total tablet weight of between 150 mg and 300 mg per 100 mg of ginkgo extract, the process comprising:
(a) mixing ginkgo extract with 6.94 mg to 27.78 mg of a binder per 100 mg of ginkgo extract, with 5.56 mg to 22.22 mg of a disintegration accelerator per 100 mg of ginkgo extract, with 1.11 mg to 4.44 mg of a flow regulating agent per 100 mg of ginkgo extract, and with 0.28 mg to 1.11 mg of a mould release agent per 100 mg of ginkgo extract, to obtain a first mixture, and then compacting and comminuting the first mixture to obtain a concentrated granulate, and
(b) mixing the concentrated granulate obtained in (a) with 22.78 mg to 91.11 mg of a further binder per 100 mg of ginkgo extract, with 11.11 mg to 44.44 mg of a further disintegration accelerator per 100 mg of ginkgo extract, with 0.56 mg to 2.22 mg of a further flow regulating agent per 100 mg of ginkgo extract, and with 1.67 mg to 6.67 mg of a further mould release agent per 100 mg of ginkgo extract, to obtain a second mixture, and pressing the second mixture into a tablet,
wherein a total amount of excipients in (a) is from 13.89 mg to 55.56 mg per 100 mg of ginkgo extract and in (b) is from 36.11 mg to 144.44 mg per 100 mg of ginkgo extract; and
wherein the binder in (a) and the further binder in (b) is microcrystalline cellulose,
wherein the disintegration accelerator in (a) and the further disintegration accelerator in (b) is independently selected from the group consisting of croscarmellose sodium, carboxymethyl starch sodium, crospovidone, and sodium starch glycolate,
wherein the flow regulating agent in (a) and the further flow regulating agent in (b) is independently selected from the group consisting of highly dispersed silica and precipitated silica, and
wherein the mould release agent in (a) and the further mould release agent in (b) is independently selected from the group consisting of magnesium stearate, stearic acid, behenic acid, sodium stearyl fumarate, glycerol dibehenate, calcium behenate, and fumaric acid.

19: ne process according to claim 18, wherein the total tablet weight is between 160 mg and 200 mg per 100 mg of ginkgo extract,
wherein in (a), the ginkgo extract is mixed with 8.33 mg to 13.89 mg of the binder per 100 mg of ginkgo extract, with 6.67 mg to 11.11 mg of the disintegration accelerator per 100 mg of ginkgo extract, with 1.33 mg to 2.22 mg of the flow regulating agent per 100 mg of ginkgo extract, and with 0.33 mg to 0.56 mg of the mould release agent per 100 mg of ginkgo extract,
wherein in (b), the concentrated granulate obtained in (a) is mixed with 27.33 mg to 45.56 mg of the further binder per 100 mg of ginkgo extract, with 13.33 mg to 22.22 mg of the further disintegration accelerator per 100 mg of ginkgo extract, with 0.67 mg to 1.11 mg of the further flow regulating agent per 100 mg of ginkgo extract, and with 2.00 mg to 3.33 mg of the further mould release agent per 100 mg of ginkgo extract, and
wherein the total amount of excipients in (a) is 16.67 mg to 27.78 mg per 100 mg of ginkgo extract and in (b) is 43.33 mg to 72.22 mg per 100 mg of ginkgo extract.

20: The process according to claim 18, wherein the disintegration accelerator in (a) and the further disintegration accelerator in (b) is croscarmellose sodium,
wherein the flow regulating agent in (a) and the further flow regulating agent in (b) is precipitated silica, and
wherein the mould release agent in (a) and the further mould release agent in (b) is magnesium stearate.

21: The process according to claim 18, wherein the compacting in (a) is performed by roller compacting.

22: The process according to claim 18, wherein, apart from the ginkgo extract, no further plant extracts or other active ingredients are used, and, apart from the excipients mentioned, no further excipients are used.

23: A rapidly disintegrating tablet having a disintegration time of at most 15 minutes for peroral administration of a dry extract of leaves of Ginkgo biloba and having a total weight of the tablet between 150 mg and 300 mg per 100 mg of Ginkgo extract, prepared by the process according to claim 18.

24: The rapidly disintegrating tablet according to claim 23, wherein the tablet comprises, in addition to the ginkgo extract, microcrystalline cellulose as the binder in (a) and the further binder in (b): the disintegration accelerator in (a) and the further disintegration accelerator in (b) are independently selected from the group consisting of croscarmellose sodium, carboxymethyl starch sodium, crospovidone, and sodium starch glycolate; the flow regulating agent in (a) and the further flow regulating agent in (b) are independently selected from the group consisting of highly dispersed silica and precipitated silica; and the mould release agent in (a) and the further mould release agent in (b) are independently selected from the group consisting of magnesium stearate, stearic acid, behenic acid, sodium stearyl fumarate, glycerol dibehenate, calcium behenate, and fumaric acid,
wherein no lactose is contained in the tablet, and
wherein apart from the ginkgo extract, no other plant extracts or other active ingredients are contained in the tablet and, apart from the excipients mentioned, no other excipients are contained in the tablet.

25: A rapidly disintegrating tablet having a disintegration time of at most 15 minutes for peroral administration of a dry extract of leaves of Ginkgo biloba,
wherein a total weight of the tablet is between 150 mg and 30) mg per 100 mg of ginkgo extract,
wherein the tablet comprises, in addition to the ginkgo extract, microcrystalline cellulose as a binder; a disintegration accelerator selected from the group consisting of croscarmellose sodium, carboxymethyl starch sodium, crospovidone and sodium starch glycolate; a flow regulating agent selected from the group consisting of highly dispersed silica and precipitated silica, and a mould release agent selected from the group consisting of magnesium stearate, stearic acid, behenic acid, sodium stearyl fumarate, glycerol dibehenate, calcium behenate, and fumaric acid;
wherein no lactose is contained in the tablet, and
wherein apart from the ginkgo extract, no other plant extracts or other active ingredients are contained in the tablet and, apart from excipients mentioned, no other excipients are contained in the tablet.

26: The tablet according to claim 23, wherein the total weight of the tablet is between 160 mg and 200 mg per 100 mg of ginkgo extract.

27: The tablet according to claim 23, wherein the total weight of the tablet is 175 mg per 100 mg of ginkgo extract.

28: The tablet according to claim 23, comprising 80 to 360 mg of the dry extract of leaves of Ginkgo biloba.

29: The tablet according to claim 23, comprising 220 to 260 mg of the dry extract of leaves of Ginkgo biloba.

30: The tablet according to claim 23, comprising 240 mg of the dry extract of leaves of Ginkgo biloba.

31: The tablet according to claim 23, wherein the dry extract of leaves of Ginkgo biloba comprises, by weight, 22.0 to 27.0% of flavonoids calculated as flavone glycosides, 2.6 to 3.2% of bilobalide, and 2.8 to 3.4% of ginkgolides A, B and C; and at most ppm of ginkgolic acids.

32: The tablet according to claim 23, wherein no lactose is contained in the tablet.

33: The tablet according to claim 23, wherein, apart from the ginkgo extract, no other plant extracts or other active ingredients are contained in the tablet, and, apart from the excipients mentioned, no other excipients are contained in the tablet.

34: The tablet according to claim 24, wherein the binder is microcrystalline cellulose, the disintegration accelerator is croscarmellose sodium, the flow regulating agent is precipitated silica, and the mould release agent is magnesium stearate.

35: The tablet according to claim 23, wherein the tablet is additionally coated with a film and has a disintegration time of at most 30 minutes.
